(19) United States
(12) Patent Application Publication
(10) Pub. No.: US 2002/0182184 A1
(43) Pub. Date: Dec. 5, 2002

(54) Composition for the safe removal of indoor allergens

(75) Inventors: Richard E. Pearl, Hinkley, OH (US); Scott E. Zeilinger, Pepper Pike, OH (US)

Correspondence Address: BENESCH, FRIEDLANDER, COPLAN & ARONOFF LLP, ATTN: IP DEPARTMENT DOCKET CLERK, 2300 BP TOWER, 200 PUBLIC SQUARE, CLEVELAND, OH, 44114, US

(73) Assignee: Pentagonal Holdings, Inc., Pepper Pike, OH (US)

(21) Appl. No.: 10/152,558

(22) Filed: May 21, 2002

Related U.S. Application Data
(63) Continuation of application No. 09/612,637, filed on Jul. 7, 2000.
(60) Provisional application No. 60/143,186, filed on Jul. 9, 1999.

(51) Int. Cl. A61K 38/54; A61K 38/44; A61K 38/51; A61K 38/52; A61K 38/53; A61K 38/46; A61K 38/47

(52) U.S. Cl. 424/93.4; 424/93.46; 424/94.2; 424/94.4; 424/94.5; 424/94.61; 424/94.6

(57) ABSTRACT

This invention provides for an enzymatic cleaning composition that will reduce and remove allergens and perform general cleaning at the same time. The enzymatic cleaning composition comprises an enzyme and/or a bacterial spore substance capable of producing enzymes, a wetting agent, an odor-encapsulating agent, a neutralizing agent, a surfactant-encapsulating agent, an embrittling agent and water. The enzymatic composition is applied to carpets, upholstery, drapes and other fabrics, and hard surfaces. The applied composition dries and subsequently the allergens can be removed. Additionally, the present invention provides for a new and unique manner of delivery of the enzymatic composition.

### CROSS REFERENCE TO RELATED APPLICATION

This application claims the benefit of U.S. Provisional Application serial No. 60/143,186, filed on Jul. 9, 1999.

### FIELD OF INVENTION

The present invention relates to a composition that removes allergens from the indoor environment. The composition of the present invention reduces or removes allergens in an indoor environment through an enzymatic cleaning composition and more particularly to an encapsulating enzymatic cleaning composition. In particular, the cleaning composition neutralizes and encapsulates common allergens for effective and more complete removal from the indoor environment.

### BACKGROUND OF THE INVENTION

Allergies are the country's sixth most prevalent chronic health problem. It is estimated that 20% of the U.S. population is affected by allergic rhinitis and 8% to 12% are affected by asthma. Dust in the office or home environment, or “house dust,” is a source for the troublesome symptoms of allergies for a large percentage of the population. The symptoms that result from allergies to house dust are usually those of perennial allergic rhinitis, or unusual sneezing, runny nose or nasal obstruction, and watery itching eyes. The symptoms of seasonal allergic rhinitis are identical and commonly called “hay fever.” Asthma is a disease characterized by an increased responsiveness of the bronchial tubes to various stimuli, such as allergens, cold air or exercise. The symptoms of allergic rhinitis affect the head and nasal passages and the symptoms of asthma are associated with the lungs, but the causes are similar.

An allergen is a substance that causes an exaggerated response by a person's immune system. Most allergens are complex protein molecules that contain multiple antigenic sites. The portions of a molecule that can be antigenic, called determinants, are those accessible on the surface of the molecule. All major allergens are proteinaceous material—in particular, they are glycoproteins. Glycoproteins are protein-carbohydrate complexes of a protein-polysaccharide composition, wherein the protein portion carries the chemically allergenic moiety commonly referred to as the determinant, and the polysaccharide portion forms the polymer backbone.

The harmful effects of allergens in the environment have been recognized for some time. Dog and cat hair had first been considered an allergen, however, the causative agent has since been identified as saliva enzymes on the pet dander. Fauna in dust, particularly arthropods, such as dust mites and their debris, has received attention in relation to house dust atopy, an important factor in the etiology of bronchial asthma and rhinitis. More recently, attention has been focused on outdoor allergens such as molds and pollens. Both outdoor and indoor sources of environmental allergens contribute to allergens found in the indoor environment.

Identified and characterized allergens are extremely diverse, and include cat dander and saliva (Fel d I), dog dander (Can f I), dust-mite excrement enzymes (Der p I and Der f I), cockroach parts (Bla g I, Bla g II, and Per a I), rye grass pollen (Lol p V), leaf mold spores (Penicillium, Altemaria, Aspergillus, Cladosporium), and windowsill mold spores (Aureobasidium).

In general, it is difficult to see or otherwise detect allergens because of their minute size. The majority of physical agents causing allergies are free-floating particles in the micron and sub-micron size range. All airborne allergens are subject to gravity and eventually settle. Some, depending on their aerodynamic properties, can be circulated in the air for long periods of time, before gravity acts on the allergen.

It is difficult to remove allergens from the indoor environment because of their size and properties. Today's modern HVAC (heating, ventilating and air-conditioning) systems can keep allergen particles airborne for long periods of time. Regular central furnace/air-conditioner filters are designed to capture large particulate matter that can damage heat exchangers and mechanical equipment, but typical filters allow the smaller allergens to pass through unimpeded and continue circulating throughout the living environment. Many homes and central office buildings have closed HVAC systems that, for efficiency reasons, allow very limited amounts of fresh outside air to be added to the indoor air. Some types of buildings have electric, hydronic, steam or solar heating and, hence, do not even have a central filtering system. Portable room air filters can be installed, but they are noisy, awkward, hard to maintain and inefficient. Vacuum cleaners return the filtered air to the room, and the very fine particles of allergens are redispersed into the environment. Floors can be wet-mopped and countertops wiped clean to reduce allergens, but carpet, upholstery, and window treatments such as drapes are difficult to rid of allergens because the allergens can be trapped in the pile of the fabric.

Typical methods of reducing and removing allergens from the environment include physical intervention and chemical measures. Reducing physical contact with the allergen can be partially accomplished through the elimination, separation and filtration of allergens from the environment. Chemical methods have involved the application of chemical solutions including pesticides that can linger in the environment. The long term use of strong chemical applications and exposure to them is questionable. Sufferers often resort to over-the-counter drugs and prescription drugs to reduce the symptoms of allergens. Numerous drug preparations, notably steroids, are available to control the body's response to allergies.

U.S. Pat. No. 5,843,981 discloses a method for killing dust mites and preventing associated allergies by using a mixture of pyrethrin, tetramethrin and piperonyl butoxide. U.S. Pat. No. 5,271,947 discloses a method for reducing the population of dust mites by using a sodium chloride powder. U.S. Pat. No. 4,666,940 discloses a cleaning composition for removing house mites by a compound containing a benzyl benzoate, a solid component, and a cleansing ingredient. U.S. Pat. Nos. 4,977,142 and 4,806,526 disclose an antiallergenic agent containing a tannic acid miticide composition. U.S. Pat. No. 5,569,411 discloses an aqueous liquid detergent composition for cleaning hard surfaces and repelling insects. U.S. Pat. No. 5,736,494 discloses a granular cleaning composition. U.S. Pat. No. 5,407,609 discloses a method to mircoencapsulate agents such as allergens. U.S. Pat. No. 5,672,362 discloses a method to control dust mites by using disodium octaborate tetrahydrate.

The prior art discloses different methods for killing mites but does not disclose any cleaning compositions that neutralize and encapsulate the “broad range” of allergens and, thus, safely and thoroughly removes them from the indoor environment.

### SUMMARY OF INVENTION

It is advantageous to produce a new and unique composition for the reduction of all types of allergens from the indoor environment. It is advantageous to produce a composition that performs general cleaning and removal of allergens at the same time. The invention is a new encapsulating enzymatic composition that removes allergens from the air, hard surfaces, and fabric surfaces and clumps the allergens so that they can be removed from the indoor environment by traditional methods such as vacuum cleaners and/or furnaces with standard filters and by water rinsing hard surfaces. The enzymatic composition can be applied as an aqueous solution to encapsulate all known allergens in a brittle film that dries and is easily crushed into large flakes for easy removal, thereby resulting in a clean environment. The encapsulating enzymatic composition can be applied onto fabrics such as carpets, drapes, and upholstery to clump and encapsulate the allergens, and after drying, the clumped allergens can be removed by traditional methods and equipment such as vacuuming. Further, the enzymatic composition removes the allergens from floors and other surfaces without dispersing them into the air. Further, the enzymatic composition can be used to reduce and remove allergens found on hard surfaces, HVAC systems, etc. The enzymatic composition can be added to laundry to remove the allergens in clothing, bedding and other washable items.

The present invention provides a unique encapsulating enzymatic composition that removes allergens from the indoor environment. Additionally, the present invention provides for a new and unique manner of delivery of the encapsulating enzymatic composition. The present invention provides for an encapsulating enzymatic composition comprising;

1. an enzymatic material selected, preferably selected from the group consisting of a bacterial spore substance capable of producing enzymes, an enzyme and combination thereof, wherein the enzymatic material exhibits enzymatic activity against the allergen;

2. a wetting agent;

3. an odor-encapsulating agent;

4. a neutralizing agent;

5. a surfactant-encapsulating agent;

6. an embrittling agent; and

7. water.

### DETAILED DESCRIPTION OF INVENTION

The present invention provides for an enzymatic composition that will reduce and remove allergens and perform general cleaning at the same time. The enzymatic cleaning composition comprises an enzyme material (which can include an enzyme capable of acting as an organic catalyst to break down allergens and/or a bacterial spore substance capable of producing enzymes which are capable of acting as an organic catalyst to break down allergens), a wetting agent, an odor-encapsulating agent, a neutralizing agent, a surfactant-encapsulating agent, an embrittling agent and water. The encapsulating enzymatic composition is applied to carpets, upholstery, drapes and other fabrics, and hard surfaces. The applied composition dries and subsequently the allergens are removed. The composition may clean the surface to which it is applied. The composition is nontoxic and not an eye or skin irritant. The composition contains no carcinogens, no pathogens, nothing to cause hypersensitivity, and does not otherwise pollute the indoor/outdoor environment with harmful chemicals.

The enzyme material exhibits enzymatic activity against specific proteins. There are many different types of enzymes, each one affecting a different group of compounds such as proteins, carbohydrates, fats and the like. A specific type of enzyme can cause the break down of a particular kind of compound. Enzymes break down complex materials into simpler molecules by acting as organic catalysts. After a particular series of chemical reactions is complete, the enzyme is released unchanged to take part in another reaction. Enzymes take no direct part in these chemical reactions, thus their catalytic activities can be repeated. Enzymes are derived from living organisms and as a part of living systems they perform at atmospheric pressures, mild temperatures and wide pH ranges. The useful enzymes exhibit enzymatic activity against the allergen.

The enzymes useful in the present invention include, but are not limited to, proteases, lipases, amylases, cellulases, alpha-amylases, beta-glucanases, catalases, galactomannases, glucose isomerases, glucoamylases, pectinases and combinations thereof.

Proteases are available under the names Alcalase®, Savinase®, Everlase™, and Esperase®, from Novo Nordisk; and Purafect®, Purafect® OX, and Properase® are available from Genencor. Lipases can be found under the name of Lipolase®, Lipolase Ultra, and LipoPrime™ available from Novo Nordisk. Amylases are available under the name Termamyl®, BAN, and Duramyl® from Novo Nordisk; and Purstar™ ST and Purstar OxAm are available from Genencor. Cellulase is available under the name Puradax™ HA from Genencor; and Celluzyme® and Carezyme® from Novo Nordisk. Blends of enzymatic materials are available under the names Purafect®/Purastar™, Purafect® OX/Purastar™ ST, Purafect® and OX/Purastar™ OxAm available from Genencor.

The preferred enzymes are proteases, amylases, cellulases, and the like. The enzymes are used at a concentration of about 0.01% to about 10%, and preferably about 0.1% to about 1.0% of the composition and exhibit enzymatic activity against the allergen. The enzymes can be used alone or in combination.

Any bacterial spore capable of producing enzymes that exhibit enzymatic activity against the allergens can be used in this invention. The spores useful in the present invention are from naturally occurring bacteria or from genetically altered bacteria that exhibit enzymatic activity against the allergens. The bacteria can be either anaerobic or aerobic.

The bacterial spores include but are not limited to Bacillus, Nitrobacter, Nitrosomions, Pseudomonas, Enterobacter and the like. The bacterial spores are available under the names Bi-Chem® MSB, Bi-Chem® Purta Treat, Bi-Chem® BDO, Bi-Chem® Sani-Bac®, Bi-Chem® Bio-Scrub®, Bi-Chem GC600L®, and Bi-Chem® Bioclean available from Sybron Chemical, Inc.; and Sporzyme® WC Wash, Sporzyme® Ultrasbase 2, Sporzyme® 1B and Sporzyme® FE available from Semco Laboratories Inc. The bacterial spores release enzymes that exhibit enzymatic activity against the allergen. The spores are used at a concentration of about 1.0×10⁶ to about 1.0×10⁹ CFU (colony forming unit) per milliliter of cleaning composition, and preferably about 5.0×10⁷ to about 5.0×10⁸ CFU per ml of the enzymatic composition. The bacterial spores can be used alone or in combination.

Additionally, the enzyme material or spore material can be used alone or in combination with each other as long as there is no deleterious effect on the activity of the enzymes against the allergens.

The composition of the present invention also optionally includes a wetting agent to allow the composition to penetrate and wet individual fibers or textiles. The wetting agents include but are not limited to isopropyl alcohol, ethyl alcohol, fluorosurfactants and the like. The preferred wetting agent is isopropyl alcohol. The concentration of the wetting agent is from about 0% to about 5%, and preferably about 0.5% to about 2% of the composition. The wetting agent can be used alone or in combination.

The composition of the present invention optionally includes an odor-encapsulating agent designed to physically encompass and trap and thereby neutralize any foul odor molecules. The odor encapsulating agents include but are not limited to Ordenone by Belle-Aire Fragrance, Odor Trap by U.S. Flavor and Fragrance, Flexisorb OD-100 by Innovative Chemical Technology Inc., Meelium by Prentiss Inc., Tego® Sorb Conc. 50 by Goldschmidt Chemical Corp., uncomplexed cyclodextrin, zeolites and the like. The preferred encapsulating agent is Ordenone. The concentration of the odor-encapsulating agent is from 0% to about 5%, and preferably about 0.5% to about 2% of the composition. The odor-encapsulating agents can be used alone or in combination.

The composition of the present invention also preferably includes a neutralizing agent designed to neutralize fatty acid odor causing compounds. The neutralizing agent includes but is not limited to sodium bicarbonate, sodium carbonate, sodium hydroxide, potassium hydroxide and the like. The concentration of the neutralizing agent is from about 0% to about 5%, and preferably about 0.5% to about 2% of the composition. The neutralizing agent can be used alone or in combination.

The composition of the present invention includes a surfactant-encapsulating agent to lower the surface tension of the solution and allows soap-like bubbles to form. Although the preferred embodiment of this invention utilizes a surfactant-encapsulating agent, it is understood that this invention could be utilized without using a surfactant-encapsulating agent. However, in describing the preferred embodiment, the surfactant-encapsulating agents include but are not limited to sodium lauryl sulfate, sodium lauroyl sarcosinate, amphoteric surfactants, anionic surfactants, cationic surfactants, non-ionic surfactants and the like. The surfactants include but are not limited to: amphoteric surfactants such as acyl/dialkyl ethylenediamines and derivatives, n-alkyl amino acids and the like; anionic surfactants such as acylglutamates, acylpeptides, sarconsinates, taurates and the like; carboxylic acids and salts such as alkanoic acids, ester carboxylic acids, ether carboxylic acids and the like; phosphoric acid esters and salts and the like; sulfonic acids and salts, such as acyl isethionates, alkylaryl sulfonates, alkyl sulfonates, sulfosuccinates and the like; sulfuric acid esters, such as alkyl ether sulfates, alkyl sulfates and the like; cationic surfactants, such as alkyamines, alkyl imidazolines, ethoxylated amines, quaternairies, such as alkylbenzyldimethylammonium salts, alkyl betaines, heterocyclic ammonium salts, tetraalkylammonium salts and the like; nonionic surfactants consisting of alcohols; alkanolamide such as alkanolamine-derived amides and the like; ethoxylated amides and the like; amine oxides; esters such as ethoxylated carboxylic acids, ethoxylated glycerides, glycol esters (and derivatives), monoglycerides, polyglyceryl esters, polyhydric alcohol esters and ethers, sorbitan/soribtol esters, triesters of phosphoric acid and the like; ethers such as ethoxylated alcohols, ethoxylated lanolin, ethoxylated polysiloxanes, propoxylated POE ethers, alkylpolyglycosides and the like. The preferred surfactant-encapsulating agents are sodium lauryl sulfate, sodium lauroyl sarcosinate and combinations thereof The concentration of the surfactant-encapsulating agent is about 0.1% to about 5%, and preferably about 0.5% to about 2% of the composition. The surfactant-encapsulating agent can be used alone or in combination.

The composition of the present invention optionally includes an embrittling agent to stabilize the surfactant-encapsulating film and allow it to become brittle and flaky when dry. The embrittling agents include, but are not limited to, acrylic copolymers, such as water-borne carboxylated acrylic copolymer, water-borne sodium acrylate copolymer, water-borne styrene acrylate copolymer and the like. The preferred embrittling agent is water-borne carboxylated acrylic copolymer. The concentration of the embrittling agent is about 0% to about 10%, and preferably about 0.5% to about 3% of the composition. The embrittling agent can be used alone or in combination.

The composition generally includes water to balance and usually has a near neutral or slightly alkaline pH. The water includes but is not limited to purified, deionized, or tap, and the like.

The encapsulating enzymatic composition herein can be used combined with a nontoxic glycol as an atomized spray that is sprayed toward the ceiling to seek out and weigh down airborne allergens. Gycols, such as propylene glycol or triethylene glycol, combined with the enzymatic composition, are easily atomized with mechanical action to a mist, or more finely aerosolized with a propellant or the like such as carbon dioxide and a suitable aerosol nozzle. The glycols are sticky and attach themselves to floating allergen particles, and provide weight to the particles to make them subject to gravity. Thus the allergens can fall from the air into clumps, and then can safely be removed from the indoor environment by standard furnace filters and/or a vacuum cleaners and thus not redisbursed into the air. The composition can further be diluted with water to the proper concentration prior to use and transferred to a container equipped with a trigger-type sprayer and then sprayed onto a surface. A pump or garden sprayer may be used for large applications such as a carpeted room. Any type of equipment that is capable of delivering an effective amount of the composition onto the desired surface is acceptable. “Effective amount” means the treated surface should be wet to the touch and the amount should be sufficient to visually cover the surface and thereby any allergens that have settled on the surface. The composition is sprayed lightly on hard surfaces and more heavily on fabric surfaces so as to fully wet the item being cleaned and then the item is allowed to dry. Hard surfaces may be wiped with a clean damp cloth to remove any residual material, including the encapsulated allergens and general soils. This operation also lifts spots and film from shiny surfaces. Fabric surfaces may be vacuumed immediately after drying or may be used when dry and vacuumed at a later time.

The encapsulating enzymatic composition can clean and remove allergens at the same time. The enzymatic composition can be used to remove all types of allergens found in the indoor environment and made available as an all purpose, easy to use, safe cleaning enzymatic composition.

Having generally described the invention herein, a further understanding can be obtained by reference to the specific examples that are provided herein for the purposes of illustration only, and are not intended to be limiting.

### SPECIFIC EXAMPLES

### Example 1

### Preparation of Composition

The composition is water based and produced at room temperature and pressure. A suitable container with an agitator has water in it where wetting agent, followed by the spore enzyme blend is added. After five minutes, the surfactant-encapsulating agent is added. Five minutes later the odor-encapsulating agent and embrittling agent are added followed by the neutralizing agent. After about five minutes of agitation at medium speed the composition is completely ready for use or packaging. Concentrants can be prepared in a manner similar to the above. They are simply diluted with tap water before use.

The composition was prepared using:

| | |
|---|---|
| 85.0% | Deionized water |
| 10.0% | Bi-Chem GC 600L 10X NF, by Sybron Chemicals, a multiple |
| | spore blend containing three bacterial strains of the genus |
| | Bacillus: |
| | 1. Bacillus licheniforms |
| | 2. Bacillus subtiles |
| | 3. Bacillus polymyra |
| 2.5% | Syntran DX6-54, by Interpolymer Corp, an acrylic copolymer |
| | aqueous solution |
| 1.0% | Isopropyl alcohol, a wetting agent |
| 0.5% | Ordenone, by Belle-Aire Fragrance, an odor controller |
| 0.5% | Sodium bicarbonate, a neutralizer |
| 0.5% | Sodium lauroyl sarcosinate, 30% aqueous, a mild synthetic |
| | detergent |

The composition has 5.4×10⁷ CFU per milliliter of cleaning composition. A one-square yard piece of five-year-old carpet was removed from a bedroom floor near a window of a non-air-conditioned home that maintains a cat and dog as pets. The carpet is contaminated with various amounts of dust mite and other animal products, pollen, mold and fungi. A volunteer with diagnosed allergies placed the carpet in his bed with his pillow on top and slept on the carpet for eight hours one night, and awoke the next morning with complaints of inflamed allergies. The carpet was then treated with 4 ounces of the composition and allowed to air dry for 8 hours followed by a thorough vacuuming. The volunteer whose allergy symptoms had subsided after 16 hours, then placed the treated carpet in his bed with pillow on top and slept on the carpet another night with no complaints of allergies the next morning. The allergens on the carpet had been neutralized and removed, as evidenced by the exposure experiment.

### Example 2

### Preparation of Composition

The composition is water based and produced at room temperature and pressure. A suitable container with an agitator has water in it, where a spore enzyme blend is added. After five minutes the wetting agent and surfactant-encapsulating agents are added. Five minutes later the embrittling agent is added. After about five minutes of agitation at medium speed, the composition is completely ready for use and transferred to a plastic spray bottle equipped with a trigger sprayer.

The composition was prepared, using:

| | |
|---|---|
| 70.0% | Deionized water |
| 25.0% | Bi-Chem Bioclean 4X, by Sybron Chemicals, a blend of Bacillus |
| | spores containing: |
| | 1. Bacillus licheniformis |
| | 2. Bacillus amyloliquifaciens |
| | 3. Bacillus pasteurii |
| | 4. Bacillus laevolacticus |
| 3.0% | Syntran 40-15 |
| 1.0% | Sodium lauryl sulfate, 30% aqueous |
| 1.0% | Ethanol |

The composition has 5.4×10⁷ CFU per milliliter of cleaning composition. The composition was sprayed onto the lower portion of an insulated glass window and adjoining window sill that were contaminated with black mold and allowed to dry. The next day, the black mold was modified to a light brown color and easily wiped away with a damp cloth, leaving a mold-free window and sill. The allergens that could be released by the spore-producing mold were removed from the indoor environment.

### Example 3

### Preparation of Composition, Toxicity Test

The composition is water-based and produced at room temperature and pressure. A suitable container with an agitator contains the water. A wetting agent is added to the water followed by a spore enzyme blend. After about five minutes, the surfactant-encapsulating agents are added. About five minutes later the odor-encapsulating agent is added, followed by the embrittling agent. After about five minutes of agitation at medium speed, the composition is completely ready for use and transferred to a plastic spray bottle equipped with a trigger sprayer.

The composition was prepared using:

| | |
|---|---|
| 83.0% | Deionized water |
| 10.0% | Bi-Chem MSB 10X NF, by Sybron Chemical, Inc., a multiple |
| | spore blend of Bacillus spores, containing: |
| | 1. Bacillus licheniformis |
| | 2. Bacillus amyloliquifaciens |
| | 3. Bacillus pastuerii |
| | 4. Bacillus laevolacticus |
| 2.0% | Sodium lauryl sulfate, 30% aqueous |
| 2.0% | Isopropyl alcohol |
| 2.0% | Syntran 40-15 |
| 0.5% | Sodium lauroyl sarcosinate, 30% aqueous |
| 0.5% | Flexisorb OD-100 |

The composition has 5.4×10⁷ CFU per milliliter of cleaning composition.

The multiple bacterial spore solution (“MSB”) was tested by an independent laboratory for Delayed Contact Hypersensitivity by the Modified Buehler Method at the same concentration as the cleaning composition. The conclusion was it would not be considered a sensitizer in albino guinea pigs. The guinea pig is the preferred species for the determination of sensitization potential. Studies using the guinea pig are in compliance with recommendations of the FDA, EPA and CPSC.

The MSB multiple bacterial spore solution was further tested for Acute Oral Toxicity by 40 CFR Subpart B, 798.1175 at the same concentration as the cleaning composition, and found to meet the labeling requirements for Toxicity Category III as defined by 40 CFR 156.10(c)(2)(i) or essentially non-toxic.

The MSB multiple bacterial spore solution was further tested for Acute Inhalation Toxicity at the same concentration as the cleaning composition in accordance with the Pesticide Assessment Guidelines, Subdivision F: Human and Domestic Animals, Series 81-3. It was concluded that, since the LC₅₀ is greater than 2.75 mg/liter, it meets the criteria for Toxicity Category III, as defined in 40 CFR 156.10 or essentially non-toxic.

The composition of this example was sprayed on a raised, light gray, fluffy, downy mildew on the outside wall of an unheated closet that is normally kept pitch black. A similar patch of mildew, about two feet away on the same wall, was treated by the same method using only distilled water. Seventy-two hours later, the mildew treated with the cleaning composition was flat on the wall and cream-colored. The mildew treated with the water was still fluffy and downy and light gray. A damp cloth would remove the downy part of the mildew treated with water, but the base of the mildew was still tenaciously attached to the wall surface. A damp cloth easily and completely removed the mildew that was treated with the cleaning composition, resulting in an allergen-free surface.

### Example 4

### Preparation of Composition

The composition is water-based and produced at room temperature and pressure. A suitable container with an agitator has water added and then the spore enzyme blend is added. After about five minutes the wetting agent and surfactant encapsulation agents are added. About five minutes later the embrittling agent is added. After about five minutes of agitation at medium speed, the composition is completely ready for use and transferred to a one-gallon garden-type sprayer.

The composition was prepared using:

| | |
|---|---|
| 86.00% | Deionized Water |
| 10.00% | Sporzyme Ultrabase 2, by Semco Laboratories, Inc., a multiple |
| | spore blend of Bacillus spores, containing: |
| | 1. Bacillus licheniformis |
| | 2. Bacillus amyloliquifaciens |
| | 3. Bacillus subtillis |
| | 4. Bacillus megaterium |
| | 5. Bacillus pumulius |
| 1.25% | Sodium lauryl sulfate, 30% aqueous |
| 1.00% | Isopropyl alcohol |
| 1.00% | Syntran 40-15 |
| 0.75% | Sodium lauroyl sarcosinate, 30% aqueous |

The composition has 6.7×10⁷ CFU per milliliter of cleaning composition. A room, approximately 8.5 by 12.5 feet, with about a 9.0 foot ceiling, has been actively used for more than five years by two long-haired domestic cats, for playing, sitting, and sleeping. It adjoins a room that contained their litter box. The room is now isolated from the cats; all furniture, wall hangings, and throw rugs are removed from the room. The only window in the room is completely sealed with 6-mil, clear PVC plastic; the only door is weatherstripped to seal it from airflow and kept closed; the only HVAC duct is sealed with duct tape. The HVAC has been turned off for the season as heat is no longer needed, and the air conditioning is not yet needed. A minimum-maximum thermometer indicates the room maintains 70° F.±5°, and the relative humidity is 50%±20%. The one-piece vinyl floor is swept of debris, but the painted woodwork trim, and vinyl-wallpapered plaster walls and ceiling are left untouched. The room is left sealed and undisturbed for 24 hours.

Day One: A volunteer with medically diagnosed cat allergies is allowed to enter the room with a book, drinking water, and a clean metal folding chair, and the door is sealed behind him. In less than ten minutes, the volunteer complains of allergy symptoms—his eyes become dry, and his nose becomes stuffed. The symptoms increase, and in less than an hour, he complains his eyes “feel” swollen. He must breathe through his mouth because his nasal passages have completely shut. Before the end of two hours, the volunteer asks to leave the room. His throat is dry and cannot be quenched by water; breathing is difficult; he sneezes periodically and his eyes are bloodshot.

The volunteer is taken outdoors where, within ten minutes, he feels some relief from his allergies. He describes the conditions in the room as “unbearable.” He states that this is his typical allergic reaction to cats when his body is free of any medication to guard against this condition. He indicates that he has taken no medication for allergies during the last 60 days. After two hours in the fresh air, his breathing passages and eyes return to normal, and he is comfortable again.

The room is treated with the anti-allergen cleaning composition of Example 4, by a worker with no history of cat allergies. The composition is lightly misted on the ceiling, walls, door, floor, and metal chair, and left to dry. The worker reports no ill effects from misting approximately 64 fluid ounces of the anti-allergen cleaning composition in the closed room. The room is left sealed and undisturbed for 72 hours.

Day Four: The same volunteer is allowed to enter the room with a book and drinking water. After five hours, he reports that he is still comfortable and has no cat allergy symptoms. He is allowed to leave the room and is interviewed outdoors. He indicates that he experienced none of the symptoms of cat allergies while in the room this time, and is not currently taking any medication for allergies.

The worker enters the room with two five-gallon pails of water and two Turkish towels, and completes two successive water rinses of the vinyl-covered ceiling, walls, woodwork, door, floor, and metal chair. The first rinse-water is sudsy and murky brown in color. The second rinse is not sudsy, and nearly clear with a light brown color. The room is left sealed and undisturbed for 24 hours.

Day Five: The same volunteer is allowed to enter the room again with a book and drinking water. After five hours, he again reports that he is still comfortable with respect to cat allergy symptoms, and again indicates he is not currently taking any medication for allergies.

The anti-allergen cleaning composition was successfull in neutralizing the cat and other common allergens that had built up in the room over a period of five years. After neutralizing, the cat allergens along with general dirt and grime can be removed from the room with a simple water rinse. The example demonstrates a method for neutralizing and removing common allergens from a domestic dwelling as well as the use of the cleaning composition therefore.

While various embodiments of an enzymatic cleaning composition for the removal of common allergens from the indoor environment have been disclosed, it should be understood that modifications and adaptations thereof will occur to persons skilled in the art. Other features and aspects of this invention will be appreciated by those skilled in the art upon reading and comprehending this disclosure. Such features, aspects, and expected variations and modifications of the reported results and examples are clearly within the scope of the invention where the invention is limited solely by the scope of the following claims.

Having thus defined the invention, we claim:

1. A composition for the removal of allergens, the composition comprising:
at least one enzyme material capable of acting as an organic catalyst to break down allergens and a surfactant-encapsulating agent to lower the surface tension of the solution and allow soap-like bubbles to form, thereby trapping said allergens and any parts thereof.

2. The composition of claim 1 wherein said at least one enzyme material is an enzyme capable of acting as an organic catalyst to break down allergens.

3. The composition of claim 1 wherein said at least one enzyme material is a bacterial spore capable of producing enzymes capable of acting as an organic catalyst to break down allergens.

4. The composition of claim 1 wherein said enzyme material comprises at least one enzyme and at least one bacterial spore capable of producing enzymes.

5. The composition of claim 2 wherein said enzyme is selected from the group consisting of proteases, lipases, amylases, cellulases, alpha-amylases, beta-glucanases, catalases, galactomannases, glucose isomerases, glucoamylases, pectinases, blends of enzymes, or combinations thereof.

6. The composition of claim 3 wherein said bacterial spore is selected from the group consisting of Bacillus, Nitrobater, Nitrosomions, Pseudomonas, Enterobacter, blends of spores, or combinations thereof.

7. A composition for the removal of allergens, the composition comprising:
a. at least one enzyme material capable of acting as an organic catalyst;
b. a wetting agent to allow the composition to penetrate and wet the treated surface;
c. an odor-encapsulating agent to physically encompass and trap and thereby neutralize foul odor molecules;
d. a neutralizing agent to neutralize fatty acid odor causing compounds;
e. a surfactant-encapsulating agent to lower the surface tension and allow soap-like bubbles to form; and
f. an embrittling agent to stabilize the surfactant-encapsulating bubbles and allow said bubbles to become brittle and flaky when dry.

8. The composition of claim 7 wherein said enzyme material is an enzyme which breaks down allergens.

9. The composition of claim 8 wherein said enzyme is selected from the group consisting of proteases, lipases, amylases, cellulases, alpha-amylases, beta-glucanases, catalases, galactomannases, glucose isomerases, glucoamylases, pectinases, blends of enzymes, or combinations thereof.

10. The composition of claim 7 wherein said enzyme material is a bacterial spore capable of producing enzymes capable of acting as an organic catalyst.

11. The composition of claim 10 wherein said bacterial spore is selected from the group consisting of Bacillus, Nitrobater, Nitrosomions, Pseudomonas, Enterobacter, blends of spores, or combinations thereof.

12. The composition of claim 8 wherein said enzyme material further comprises at least one bacterial spore capable of producing enzymes.

13. The composition of claim 12 wherein the bacterial spore material breaks down allergens and is selected from the group consisting of Bacillus, Nitrobater, Nitrosomions, Pseudomonas, Enterobacter, blends of spores, or combinations thereof.

14. The composition of claim 7 wherein the wetting agent is selected for the group consisting of isopropyl alcohol, ethyl alcohol, fluorosurfactant, or combinations thereof.

15. The composition of claim 7 wherein the wetting agent is isopropyl alcohol.

16. The composition of claim 7 wherein said odor-encapsulating agent is selected from the group consisting of uncomplexed cyclodextrin, zeolites, or combinations thereof.

17. The composition of claim 7 wherein the neutralizing agent is selected from the group consisting is sodium bicarbonate, sodium carbonate, sodium hydroxide, potassium hydroxide, or combinations thereof.

18. The composition of claim 7 wherein said neutralizing agent is sodium bicarbonate.

19. The composition of claim 7 wherein the surfactant-encapsulating agent is selected from the group consisting of sodium lauryl sulfate, sodium lauroyl sarcosinate, amphoteric surfactants, anionic surfactants, cationic surfactants, non-ionic surfactants, or combinations thereof.

20. The composition of claim 7 wherein the embrittling agent is an acrylic copolymer such as a water-borne carboxylated acrylic copolymer, a water-borne sodium acrylate copolymers, or a water-borne styrene acrylate copoylmer.

21. The composition of claim 7 wherein the embrittling agent is a water-borne carboxylated acrylic copolymer.

22. A composition for the removal of allergens, the composition comprising:
a. at least one bacterial spore capable of producing enzymes capable of acting as an organic catalyst;
b. a wetting agent to allow the composition to penetrate and wet the treated surface;
c. an odor-encapsulating agent to physically encompass and trap and thereby neutralize foul odor molecules;
d. a neutralizing agent to neutralize fatty acid odor-causing compounds;
e. a surfactant-encapsulating agent to lower the surface tension of the solution and allow soap-like bubbles to form; and
f. an embrittling agent to stabilize the surfactant-encapsulating bubbles and allow it to become brittle and flaky when dry.

23. The composition of claim 22 wherein the bacterial spore capable of breaking down allergens is selected from the group of Bacillus, Nitrobater, Nitrosomions, Pseudomonas, Enterobacter, blends of spores, or combinations thereof.

24. A composition for the removal of allergens, the composition comprising:
a. from about 0.01% to about 10% of the composition an enzyme material selected from the group consisting of enzymes capable of acting as an organic catalyst to break down allergens, bacterial spores capable of producing enzymes capable of acting as an organic catalyst to break down allergens, and combinations thereof;
b. from about 0% to about 5% of a wetting agent;
c. from about 0% to about 5% of an odor-encapsulating agent;
d. from about 0% to about 5% of a neutralizing agent;
e. from about 0% to about 5% of a surfactant-encapsulating agent;
f. from about 0% to about 10% of an embrittling agent; and
g. water at about 80% to about 99%, of the final composition.

25. The composition of claim 24 wherein the bacterial spores are used at a concentration in the range of about 1.0×10⁶ to about 1.0×10⁹ CFU per milliliter in the final composition.

26. A process to remove allergens from the indoor environment comprising:
a. preparing an encapsulating enzymatic composition comprising an enzyme material selected from the group consisting of enzymes, bacterial spores capable of producing enzymes, or combinations thereof;
b. means for delivering the encapsulating enzymatic composition onto a surface containing allergens; and
c. means for removing the composition, encapsulated allergens and parts thereof, general solids, or combinations thereof from said surface.

27. A process according to claim 26 wherein said means for delivering the encapsulating enzymatic composition comprises spraying said composition onto said surface.

28. A process according to claim 26 wherein the means for removing said composition, encapsulated allergens and parts thereof, general solids, or combinations thereof from said surface comprises the use of a vacuum sweeper.

29. A process according to claim 26 wherein the means for removing said composition, encapsulated allergens and parts thereof, general solids, or combinations thereof from said surface comprises wiping said composition from said surface.

30. A process to remove allergens from clothing, said process comprising:
a. applying an encapsulating enzymatic composition comprising an enzyme material selected from the group consisting of enzymes, bacterial spores capable of producing enzymes, or combinations thereof to clothing;
b. means for removing said encapsulating enzymatic composition, encapsulated allergens, general solids or combinations thereof from said clothing.

31. A process according to claim 30 wherein the means for applying said composition to said clothing comprises applying an amount of said composition to laundry; and
wherein the means for removing said composition, encapsulated allergens, general solids, or combinations thereof from said clothing comprises allowing a washing machine to perform its wash and rinse cycles.

32. A process to remove allergens from the air, said composition comprising:
a. preparing an enzymatic composition comprising an enzyme material selected from the group consisting of enzymes, bacterial spores capable of producing enzymes, or combinations thereof, and
b. means for delivering the encapsulating enzymatic composition into air containing airborne allergens.

33. A process according to claim 32 wherein said composition is combined with a non-toxic glycol to produce an atomized spray capable of being sprayed in the air to seek out and weigh down airborne allergens.
